(19) United States
(12) Patent Application Publication
(10) Pub. No.: US 2024/0139007 A1
(43) Pub. Date: May 2, 2024

(54) INTEGRATED DEPLOYMENT SYSTEM (DELIVERY MECHANISM) FOR INTRACRANIAL ATHEROSCLEROSIS STENT

(71) Applicant: Kai Medtech LLC, Lake Forest, CA (US)

(72) Inventors: Linh Dinh, Lake Forest, CA (US); Joshua Benjamin, Lake Forest, CA (US)

(73) Assignee: Kai Medtech LLC, Lake Forest, CA (US)

(21) Appl. No.: 18/380,193

(22) Filed: Oct. 15, 2023

Related U.S. Application Data
(60) Provisional application No. 63/416,467, filed on Oct. 14, 2022.

(51) Int. Cl. A61F 2/958; A61M 25/10

(52) U.S. Cl. CPC A61F 2/958; A61M 25/1025; A61F 2002/9583

(57) ABSTRACT

A stent delivery system comprising a balloon catheter having a catheter shaft with a balloon mounted on a distal end with a self-expanding stent positioned on the catheter shaft proximal to the balloon (tandemly) with a delivery sheath covering the self-expanding stent. In use, the stent delivery system is advanced on the guidewire to the target lesion and the balloon is inflated to dilate the lesion. The balloon is deflated and advanced distally past the lesion until the self-expanding stent positioned proximate the lesion. The sheath is withdrawn, releasing the self-expanding stent to expand and contact the lesion. Once the self-expanding stent has expanded, the stent delivery system and guidewire are withdrawn from the lesion.

### CROSS-REFERENCE TO RELATED APPLICATIONS

This application claims the benefit of U.S. Provisional Application No. 63/416,467 filed Oct. 14, 2022, which is incorporated herein by reference.

### FIELD

The present invention relates generally to the field of neurovascular surgery, and more specifically, to a stent delivery system for delivering intracranial stents for a neuro-interventional procedure.

### BACKGROUND

Atherosclerosis diseases. The devices used to treat this disease include guidewire, balloon catheter, a stent and its delivery stem. There isn't a consistent way of treating this disease, varying from patient to patient and from doctors' discretion. But the current clinical practice most widely utilized would be to dilate the disease lesion with a balloon prior to deploying a stent at the target lesion/artery location. Then use the balloon subsequent to stent placement within the stent and vessel wall again to ensure the stent apposition against the vessel wall. This procedure is time consuming because it requires the balloon catheter and stent delivery system to be exchange in and out of the body. Further challenges are due to tortuosity and vessel size and recrossing the stented area and not entangling the guidewire or balloon catheter.

Accordingly, there is a need for an improved system and method of delivering and detaching an implant to a target site or location within the neurovascular space/body of patient. The present invention is directed toward systems and methods for stent delivery system

### SUMMARY

This invention presents a way to deliver the stent to the patient with the shortest procedural times, least opportunity for wire entanglement, and ensure accurate and fully dilated stent and diseased artery. The new method also includes guidewires, balloon catheter and stent all in one system with the low crossing profile while eliminating the need for catheter exchanges.

The present invention presents method of integrating a balloon catheter as the delivery system for the self-expanding stent with guidewire support into one system for delivering intracranial stents for neuro-interventional procedure like intracranial atherosclerosis.

Embodiments of the present invention are directed to a stent delivery system comprising a balloon catheter having a catheter shaft with a balloon mounted on a distal end with a self-expanding stent positioned on the catheter shaft proximal to the balloon (tandemly) with a delivery sheath covering the self-expanding stent. In use, the stent delivery system is advanced on the guidewire to the target lesion and the balloon is inflated to dilate the lesion. The balloon is deflated and advanced distally past the lesion until the self-expanding stent positioned proximate the lesion. The sheath is withdrawn, releasing the self-expanding stent to expand and contact the lesion. Once the self-expanding stent has expanded, the balloon can move back to post dilate the stent vessel for better wall apposition, then the stent delivery system (the balloon and sheath) and guidewire are withdrawn from the lesion.

### BRIEF DESCRIPTION OF THE DRAWINGS

FIG. 1 shows one embodiment of a stent delivery system.

FIG. 2 shows the balloon on the catheter shaft in a low profile deflated state for delivery through a guide catheter.

FIG. 3A shows the self-expanding stent in a collapsed state on the catheter shaft proximal to the balloon (the sheath has been pulled back for clarity).

FIG. 3B shows the self-expanding stent expanding as the sheath is moved proximally.

FIG. 4 shows a stent delivery system that has been advanced on a guidewire through the neurovascular to a lesion site.

FIG. 5 shows the balloon has been inflated to contact the lesion to expand and dilate the lesion.

FIG. 6 shows the balloon advanced distally beyond the dilated lesion until the self-expanding stent is positioned within the dilated lesion.

FIG. 7 shows the sheath has moved proximally to release the self-expanding stent.

FIG. 8 shows the balloon post dilating the self-expanding stent.

FIGS. 9-11 show another embodiment of a stent delivery system that is advanced through the vasculature to open a target lesion

### DETAILED DESCRIPTION

The present invention describes systems and methods using a stent delivery system having a balloon and self-expanding stent mounted tandemly on the proximal end of a balloon catheter. A delivery sheath covers the self-expanding stent during delivery to a desired location.

The stent delivery system disclosed herein will minimize the risk to the patient by eliminating a balloon catheter/stent delivery catheter exchange and provide constant access to the lesion. This procedure would furthermore reduce the likelihood of vasospasm occurring during the procedure and also shortening the amount of time during the procedure.

In use, the stent delivery system is advance on a guidewire to a target lesion. The balloon is inflated to dilate the target lesion. The balloon is then deflated. At this point in a normal procedure, the doctor would withdraw the balloon catheter and exchange to a normal stent delivery system. The present invention does not require this time consuming exchange because the doctor advances the stent delivery system so the balloon is distal to the target lesion and the self-expanding stent is positioned at the dilated target lesion. The sheath is withdrawn, releasing the self-expanding stent to expand against the dilated lesion to hold it open. Optionally, the balloon can move back to post dilate the stent vessel for better wall apposition, the stent delivery system is then withdrawn leaving the stent in place.

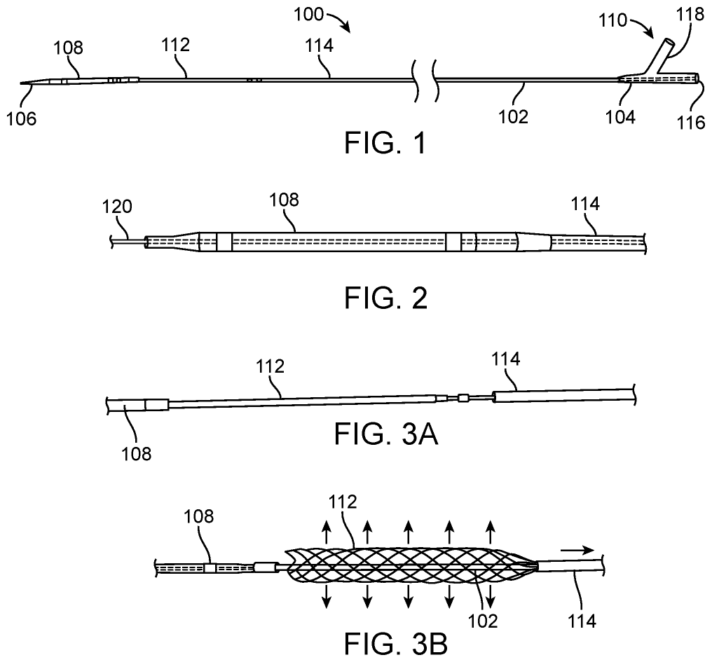

FIG. 1 shows one embodiment of a stent delivery system 100 that is advanced through the vasculature to open a target lesion. The stent delivery system 100 includes a catheter shaft 102 with a proximal end 104 and a distal end 106. An inflatable balloon 108 is coupled to the catheter shaft adjacent the distal end 106 and a connector housing 110 is coupled to the proximal end 110. A self-expanding stent 112 is positioned on the catheter shaft 102 proximal to the balloon 112 (tandemly) with a delivery sheath 114 covering the self-expanding stent 112 in a stent delivery state. The delivery sheath 114 is configured to translate proximally to a stent deployment state to uncover the stent for self-expansion.

The catheter shaft 102 includes a guidewire lumen and a balloon inflation lumen in fluid communication with the inflatable balloon 108. The connector housing 110 includes a first connector 116 coupled to the guidewire lumen and a second connector 118 coupled to the inflation lumen. The distal end of the shaft 102 includes a flexible tip 120.

FIG. 2 shows the balloon 108 on the catheter shaft in a low profile deflated state for delivery through a guide catheter. The balloon 108 is configured to expand radially when inflated with a fluid or gas to contact and expand the target lesion. Once the lesion is expanded, the fluid or gas is withdrawn the balloon 108 returns to the low profile deflated state.

FIG. 3A shows the self-expanding stent 112 in a collapsed state on the catheter shaft proximal to the balloon 108 (the sheath has been pulled back for clarity). The self-expanding stent is made of a self-expanding material, such as Nitinol, and is held in the collapsed state during delivery by the sheath 114. The sheath extends coaxially over the catheter shaft 102 with a distal end covering the self-expanding stent 112 and a proximal end near the connector housing 110.

FIG. 3B shows the self-expanding stent 112 is an expanded state. When the self-expanding stent is at the desired location, the sheath 114 is configured to slide proximally along the catheter shaft 102 to uncover the self-expanding stent 112. Once uncovered, the self-expanding stent 112 expands away from the catheter shaft from the collapsed state to the expanded state.

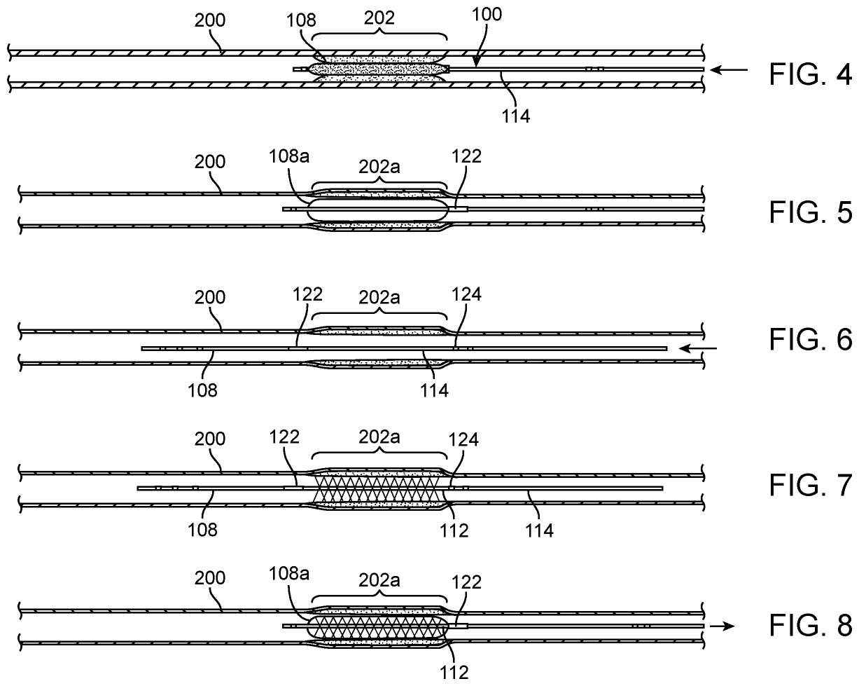

FIG. 4 shows a stent delivery system 100 that has been advanced on a guidewire 126 through the neurovascular 200 to a lesion site 202. The balloon 108 is positioned within the lesion 202. A balloon marker 122 may be used to assist in positioning the balloon.

FIG. 5 shows the balloon 108a has been inflated to contact the lesion to expand and dilate the lesion 202a. Once the lesion is dilated, the balloon 108 is deflated.

FIG. 6 shows the balloon 108 advanced distally beyond the dilated lesion 202a until the self-expanding stent 112 is positioned within the dilated lesion 202a. The balloon marker 122 and a stent marker 124 may be used to assist in positioning the self-expanding stent 112.

FIG. 7 shows the sheath 114 has moved proximally to release the self-expanding stent 112a. Once released, the self-expanding stent 112a expands to contact the dilated lesion 202a.

FIG. 8 shows the inflatable balloon 108 post dilating the self-expanding stent 112a. If the self-expanding stent 112 does not dilate the lesion 202 or only partially dilates the lesion 202, the inflatable balloon 108 can move back to lesion 202 and reinflate to post dilate and expand the self-expanding stent 312 in the vessel for better wall apposition.

The inflatable balloon 108 is then deflated and the stent delivery system 100 is withdrawn 113 proximally. In some cases, the self-expanding stent 312 does dilate the lesion 202a and the inflatable balloon 308 is not needed. The stent delivery system 300 is withdrawn 313 from lesion 202a leaving the self-expanding stent 112a to keep the lesion dilated

In some embodiments, the stent delivery system 100 is advanced to a second lesion to use the inflatable balloon 108 to dilate the second lesion.

In one embodiment, the stent delivery system is pre-assembled with the balloon and self-expanding stent positioned tandemly on the catheter shaft.

In another embodiment, the self-expanding stent and delivery sheath are combined with a balloon catheter, with that the self-expanding stent mounted on the catheter shaft proximal to the balloon catheter (tandemly) with the delivery sheath to cover the stent, as a “System”. The doctor can advance the “System” on a guidewire to a target lesion as currently preformed, then using the balloon to dilate the lesion. At this point, the doctor doesn't need to withdraw the balloon and exchange to the stent delivery system (as normal procedure), but instead the doctor would advance the system further to deploy the stent as one while the balloon and guidewire stay beyond the distal lesion and stent placement. At this point the doctor may choose to post dilate the stent/lesion again to ensure the stent wall apposition to the vessel.

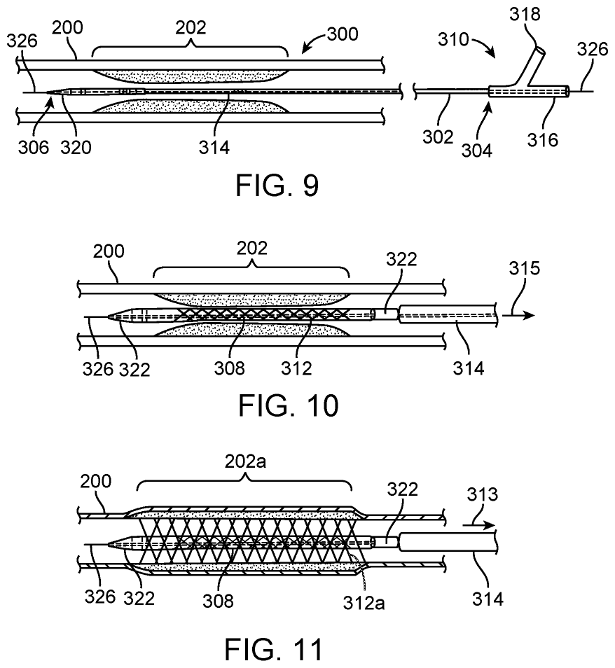

FIGS. 9-11 show another embodiment of a stent delivery system 300 that is advanced through the vasculature to open a target lesion. The stent delivery system 300 is similar to the stent delivery system 100 except that the self-expanding stent 112 mounted on the inflatable balloon 108 with the delivery sheath 114 covering both during delivery, instead of mounting the balloon and stent in tandem.

The stent delivery system 300 includes a catheter shaft 302 with a proximal end 304 and a distal end 306. An inflatable balloon 308 is coupled to the catheter shaft adjacent the distal end 306 and a self-expanding stent 312 is positioned on the inflatable balloon 308 with a delivery sheath 314 covering the self-expanding stent 312 and the inflatable balloon 308.

The catheter shaft 302 includes a guidewire lumen and a balloon inflation lumen in fluid communication with the inflatable balloon 308. The connector housing 310 includes a first connector 316 coupled to the guidewire lumen and a second connector 318 coupled to the inflation lumen. The distal end 304 of the shaft 302 includes a flexible tip 320.

FIG. 9 shows a stent delivery system 300 that has been advanced on a guidewire 320 through the neurovascular 200 to a lesion site 202. The delivery sheath 314 covering the self-expanding stent 312 and the inflatable balloon 308 are positioned within the lesion 202. A balloon marker 322 may be used to assist in positioning the self-expanding stent 312 and the inflatable balloon 308.

FIG. 10 shows the sheath 314 has moved proximally 315 to expose the self-expanding stent 312 and inflatable balloon 308.

FIG. 11 shows the released self-expanding stent 312a expands to contact and dilate the lesion 202a.

If the self-expanding stent 312 does not dilate the lesion 202, the inflatable balloon 308 can inflate to dilate and expand the self-expanding stent 312 in the vessel for better wall apposition and dilate the lesion 202 at the same time. The inflatable balloon is then deflated and the stent delivery system 300 is withdrawn 313 proximally. In some cases, the self-expanding stent 312 does dilate the lesion 202a and the inflatable balloon 308 is not needed. The stent delivery system 300 is withdrawn 313 from lesion 202a leaving the self-expanding stent 112a to keep the lesion dilated and advanced to a different lesion 204 to use the inflatable balloon 308 to dilate the second lesion 204a.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A stent delivery system comprising:
a catheter shaft having a guidewire lumen and a balloon inflation lumen with a proximal end and a distal end;
an expandable balloon coupled to the catheter shaft adjacent the distal end; the expandable balloon being fluidly coupled to the balloon inflation lumen;
a self-expanding stent positioned on the catheter shaft proximal to the balloon (mounted tandemly); and
a delivery sheath covering the self-expanding stent being configured to translate proximally from a stent delivery state covering the self-expanding stent to a stent deployment state uncovering the self-expanding stent for self-expansion.

2. The stent delivery system of claim 1, wherein the self-expanding stent is made from nitinol.

3. The stent delivery system of claim 1, further comprising a connector housing having a first connector coupled to the guidewire lumen and a second connector coupled to the inflation lumen.

4. The stent delivery system of claim 1, further comprising a balloon marker to assist in positioning the balloon.

5. The stent delivery system of claim 1, further comprising a stent marker to assist in positioning the self-expanding stent.

6. The stent delivery system of claim 1, wherein the self-expanding stent is positioned on the catheter shaft in a collapsed state for delivery to a lesion site.

7. The stent delivery system of claim 6, wherein in the uncovered state, the self-expanding stent is configured to self-expand from the collapsed state to an expanded state at the lesion site to dilate a lesion.

8. The stent delivery system of claim 1, wherein the expandable balloon is in a deflated state for delivery to a lesion.

9. The stent delivery system of claim 8, wherein the expandable balloon is configured to expand radially when inflated to contact and expand the lesion and/or post dilate the self-expanding stent into the lesion for better wall apposition.

10. A stent delivery system comprising:
a catheter shaft having an expandable balloon on a distal end configured to inflate and deflate one or more times;
a self-expanding stent positioned on the catheter shaft in a collapsed state proximal to the balloon, the self-expanding stent configured to expand from the collapsed state to an expanded state at a lesion site; and
a delivery sheath covering the self-expanding stent in the collapsed state on the catheter shaft, the delivery sheath being configured to translate proximally to uncover the self-expanding stent to self-expand to the expanded state.

11. The stent delivery system of claim 10, wherein the expandable balloon is configured to:
inflate to dilate a lesion,
deflate after the lesion is dilated,
inflate to post dilate the self-expanding stent in the lesion, and
deflate to be removed from the lesion.

12. The stent delivery system of claim 10, wherein the catheter shaft includes a guidewire lumen and a balloon inflation lumen coupled to the expandable balloon.

13. The stent delivery system of claim 12, further comprising a connector housing having a first connector coupled to the guidewire lumen and a second connector coupled to the inflation lumen.

14. The stent delivery system of claim 10, wherein the self-expanding stent is made from nitinol.

15. The stent delivery system of claim 10, further comprising a balloon marker to assist in positioning the expandable balloon.

16. The stent delivery system of claim 10, further comprising a stent marker to assist in positioning the self-expanding stent.

17. A method of using a stent delivery system comprising:
providing a stent delivery system comprising:
a catheter shaft having an expandable balloon on a distal end, the balloon configured to inflate and deflate one or more times;
a self-expanding stent positioned on the catheter shaft proximal to the balloon in a collapsed state, the self-expanding stent configured to expand from the collapsed state to an expanded state; and
a delivery sheath covering the self-expanding stent in the collapsed state on the catheter shaft, the delivery sheath being configured to translate proximally to uncover the self-expanding stent to self-expand to the expanded state;
advancing the stent delivery system through the neurovascular to a lesion site;
positioning the expandable balloon at the lesion site;
expanding the expandable balloon to dilate the lesion;
deflating the expandable balloon;
advancing the stent delivery system distally;
position the self-expanding stent in the dilated legion;
withdrawing the delivery sheath to uncover the self-expanding stent;
self expanding the uncovered self-expanding stent;
withdrawing the stent delivery system to position the expandable balloon at the dilated the self-expanding balloon;
inflating the expandable balloon to post dilate the self-expanding stent into the lesion for better wall apposition; and
deflating the expandable balloon;
removing the stent delivery system from the neurovascular.

18. The stent delivery system of claim 17, wherein the self-expanding stent is made from nitinol.

19. The stent delivery system of claim 17, further comprising a balloon marker to assist in positioning the balloon.

20. The stent delivery system of claim 17, further comprising a stent marker to assist in positioning the self-expanding stent.
